(19) United States
(12) Patent Application Publication
(10) Pub. No.: US 2020/0345213 A1
(43) Pub. Date: Nov. 5, 2020

(54) TUBE BODY AND ENDOSCOPE

(71) Applicant: OLYMPUS CORPORATION, Tokyo (JP)

(72) Inventor: Yuki MITAMURA, Tokyo (JP)

(73) Assignee: OLYMPUS CORPORATION, Tokyo (JP)

(21) Appl. No.: 16/884,617

(22) Filed: May 27, 2020

Related U.S. Application Data
(63) Continuation of application No. PCT/JP2018/033264, filed on Sep. 7, 2018.

(30) Foreign Application Priority Data
Nov. 30, 2017 (JP) 2017-230646

(51) Int. Cl. A61B 1/00

(52) U.S. Cl. CPC A61B 1/00154

(57) ABSTRACT

A tube for insertion device arranged on an outer periphery of an insertion section in an insertion device configured to be inserted into a subject and rotatable around a longitudinal axis of the insertion section includes a tapered portion formed in a distal end portion in the insertion section and having an outer diameter decreasing toward a distal end of the insertion section, and a stiffening portion provided at a distal end of the tapered portion and having a higher rigidity than a rigidity of the distal end of the tapered portion.

### CROSS REFERENCE TO RELATED APPLICATION

This application is a continuation application of PCT/JP2018/033264 filed on Sep. 7, 2018 and claims benefit of Japanese Application No. 2017-230646 filed in Japan on Nov. 30, 2017, the entire contents of which are incorporated herein by this reference.

### BACKGROUND OF THE INVENTION

### 1. Field of the Invention

The present invention relates to a tube body arranged on an outer periphery of an insertion device to be inserted into a subject, and an endoscope.

### 2. Description of the Related Art

An insertion device including an insertion section having flexibility to be inserted into a subject to observe and treat within the subject of a living body, a structure, or the like has been used in the medical field and the industrial field, for example. The insertion device includes an endoscope as disclosed in Japanese Patent No. 4513825, for example. As described in Japanese Patent No. 4513825, a tube covering an insertion section may be arranged on an outer periphery of an insertion section in the insertion device. The tube has flexibility, and the tube, together with the insertion section inserted inward, is bent.

As disclosed in Japanese Patent Application Laid-Open Publication No. 2016-54841, an insertion device provided with a fin having a helical shape on an outer peripheral surface of a tube covering an insertion section and capable of applying a driving force to the insertion section within a subject by rotating the tube with respect to the insertion section has been known. When the insertion device and the tube are used, a space between the insertion section and the tube is filled with a lubricant.

### SUMMARY OF THE INVENTION

A tube body according to an aspect of the present invention includes a tapered portion formed in a cylindrical shape extending in a longitudinal axis direction, formed at at least one end portion in the longitudinal axis direction of the cylindrical shape, and configured to decrease an outer diameter of the cylindrical shape toward the end portion, and a ring-shaped reinforcement portion provided to be connected to the end portion and having a higher rigidity than a rigidity of a distal end portion of the tapered portion.

An endoscope according to an aspect of the present invention includes an insertion section formed in an elongated shape extending from a hand side to a distal end side in a longitudinal direction and configured to be inserted into a subject, and a tube formed in a cylindrical shape having an inner periphery into which the insertion section penetrates and configured to be driven to rotate around the longitudinal axis with the insertion section inserted into the tube, in which the tube includes a tapered portion formed on the distal end side of the insertion section on an outer peripheral surface and having an outer diameter decreasing toward the distal end side of the insertion section, and a reinforcement portion connected to a distal end portion of the tapered portion, formed in a ring shape having an inner peripheral surface opposing an outer peripheral surface of the insertion section to contact the outer peripheral surface or opposing the outer peripheral surface with a gap, and having a higher rigidity than a rigidity of the distal end portion of the tapered portion.

### BRIEF DESCRIPTION OF THE DRAWINGS

FIG. 1 is a diagram for describing a configuration of an endoscope;

FIG. 2 is an external view illustrating a tube for insertion device according to a first embodiment as viewed in a direction perpendicular to a longitudinal axis;

FIG. 3 is a cross-sectional view in a plane including a longitudinal axis of a distal end portion of the tube for insertion device according to the first embodiment;

FIG. 4 is a cross-sectional view in a plane including a longitudinal axis of a distal end portion of a tube for insertion device according to a second embodiment; and

FIG. 5 is a cross-sectional view in a plane including a longitudinal axis of a distal end portion of a tube for insertion device according to a third embodiment.

### DETAILED DESCRIPTION OF THE PREFERRED EMBODIMENTS

Preferred embodiments of the present invention will be described below with reference to the drawings. In the drawings used for the following description, components are made to differ in scale to set a size of each of the components to a size sufficient to recognize the component on the drawings, and the present invention is not limited to only a number of the components described in the drawings, respective shapes of the components, a ratio of the respective sizes of the components, and a relative positional relationship among the components.

### First Embodiment

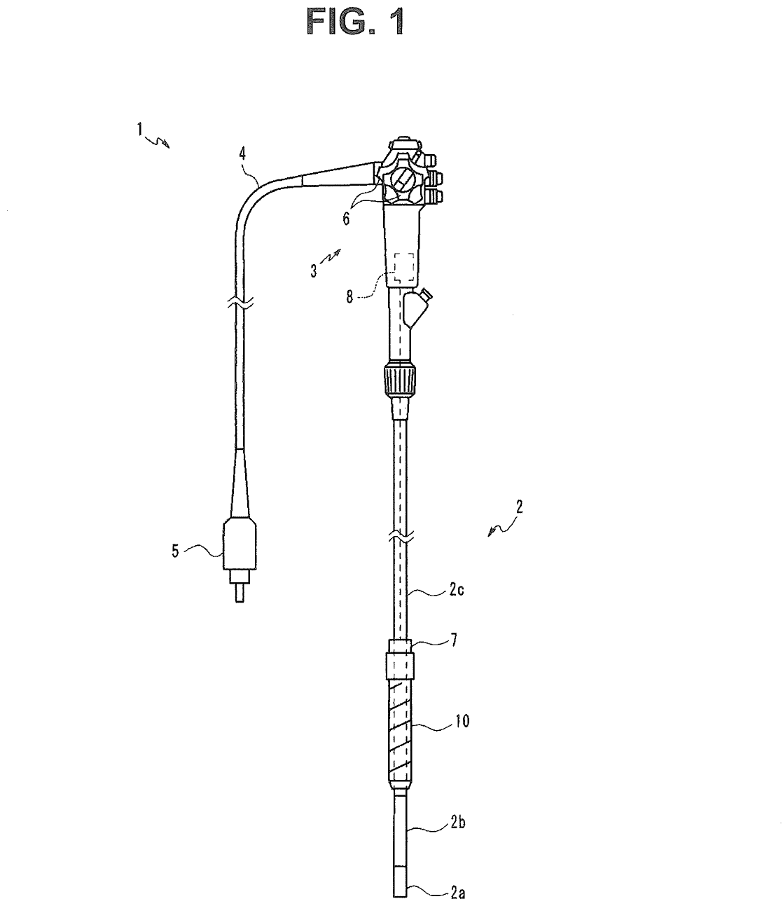

An endoscope 1 as an insertion device illustrated in FIG. 1 includes an elongated insertion section 2 configured to be able to be introduced into a subject such as a human body, and the insertion section 2 has a configuration for observing an inside of the subject. Note that the subject into which the insertion section 2 in the endoscope 1 is introduced is not limited to a human body, but may be another living body or an artifact such as a machine or a building.

The endoscope 1 according to the present embodiment mainly includes the insertion section 2 formed in an elongated shape to be introduced into the subject, an operation section 3 positioned at a proximal end of the insertion section 2, and a universal code 4 extending from the operation section 3.

The insertion section 2 is configured by consecutively providing a distal end portion 2a disposed at a distal end, a bendable bending portion 2b disposed on a proximal end side of the distal end portion 2a, and a flexible tube portion 2c having flexibility configured to connect a proximal end side of the bending portion 2b and a distal end side of the operation section 3.

A power transmission section 7 is disposed in an intermediate portion of the flexible tube portion 2c. An outer peripheral surface of the flexible tube portion 2c on a more distal end side than the power transmission section 7 has a cylindrical shape. A tube for insertion device 10 (hereinafter merely referred to as a tube), described below, arranged on an outer periphery of the flexible tube portion 2c is coupled to the power transmission section 7. The power transmission section 7 transmits motive power to be generated by an actuator 8 such as an electric motor provided in the endoscope 1 to the tube for insertion device 10. The tube 10 rotates around a longitudinal axis of the flexible tube portion 2c with respect to the flexible tube portion 2c by the motive power to be generated by the actuator 8. The actuator 8 can switch presence or absence of the generation of the motive power depending on an operation of a switch not illustrated.

The transmission of the motive power between the power transmission section 7 and the tube 10 may be in a form to be performed by coupling due to mechanical meshing between the power transmission section 7 and the tube 10, may be in a form to be performed by coupling due to a friction between the power transmission section 7 and the tube 10, or may be in a form to be performed by coupling due to a magnetic force between the power transmission section 7 and the tube 10.

A configuration for observing the inside of the subject, for example, is disposed in the distal end portion 2a. For example, an image pickup unit including an objective lens and an image pickup device and configured to optically observe the inside of the subject is disposed in the distal end portion 2a. An illumination light emission section configured to emit light for illuminating an object to be observed by the image pickup unit is also provided in the distal end portion 2a. Note that an ultrasound transducer configured to acoustically observe the inside of the subject using an ultrasound may be disposed in the distal end portion 2a.

The operation section 3 disposed at the proximal end of the insertion section 2 is provided with an angle operation knob 6 configured to operate bending of the bending portion 2b. An endoscope connector 5 configured to be connectable to an external device not illustrated is provided in a proximal end portion of the universal code 4. The external device to which the connector 5 is connected includes a camera control unit configured to control the image pickup unit provided in the distal end portion 2a, for example.

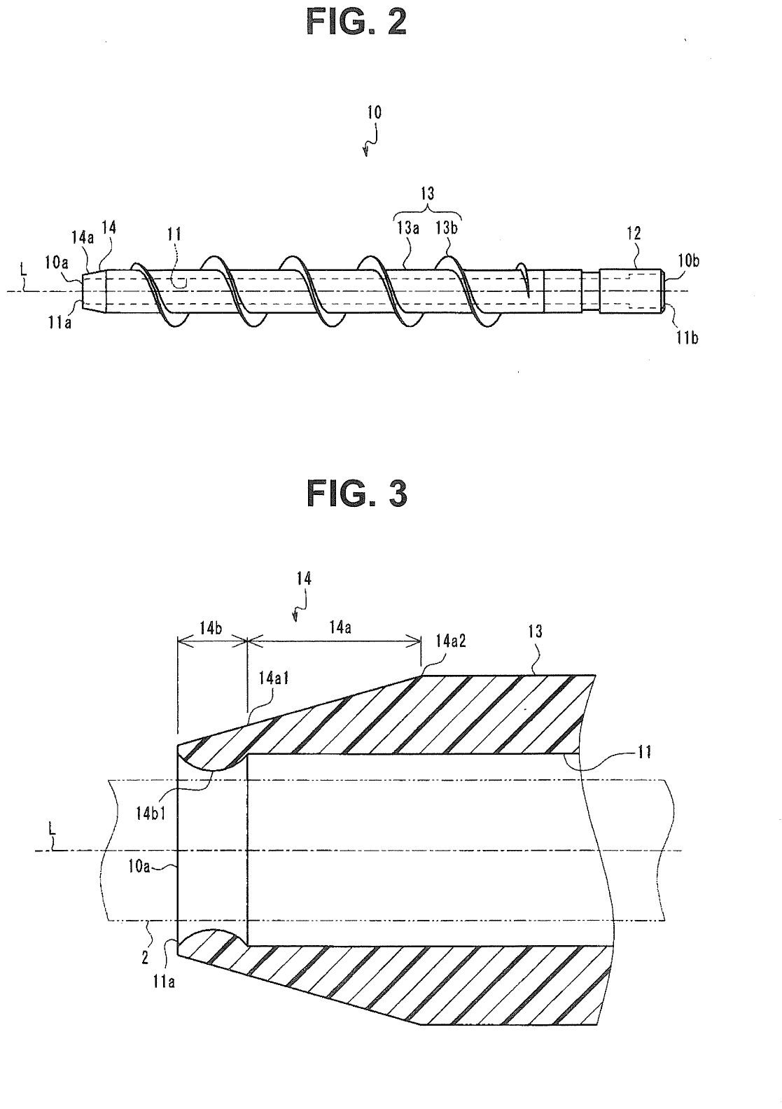

Then, a configuration of the tube 10 will be described. FIG. 2 is an external view illustrating the tube 10 viewed in a direction perpendicular to a longitudinal axis L. FIG. 3 is a cross-sectional view on a plane including the longitudinal axis L in a distal end portion of the tube 10.

The tube 10 has a cylindrical shape through which the insertion section 2 in the endoscope 1 can be inserted. A hole provided in the tube 10 and configured to allow insertion of the insertion section 2 will be hereinafter referred to as a through hole 11. One end of the tube 10 in a direction along the longitudinal axis L is referred to as a distal end 10a, and the other end of the tube 10 is referred to as a proximal end 10b. A direction toward the distal end 10a from the proximal end 10b in the direction along the longitudinal axis L is referred to as a distal end direction, and a direction opposite to the direction is referred to as a proximal end direction. In FIGS. 2 and 3, a left direction and a right direction of the drawing are respectively a distal end direction and a proximal end direction.

In the present embodiment, the longitudinal axis L of the tube 10 is a center axis of the through hole 11, and the through hole 11 opens in the distal end direction and the proximal end direction, respectively, at the distal end 10a and the proximal end 10b of the tube 10. Note that although the longitudinal axis L is drawn as a straight line in FIG. 2, an actual longitudinal axis L may have a curved shape because at least a part of the tube 10 has flexibility.

The tube 10 is obtained by consecutively connecting a connector portion 12, an intermediate cylindrical portion 13, and a distal end portion 14 to one another toward the distal end 10a from the proximal end 10b along the longitudinal axis L.

A proximal end-side opening l lb of the through hole 11 is formed in the connector portion 12. The connector portion 12 is coupled to the power transmission section 7 with the insertion section 2 in the endoscope 1 inserted into the through hole 11. In other words, the connector portion 12 is a site to which motive power to be generated by the actuator 8 is transmitted.

The intermediate cylindrical portion 13 has flexibility. Therefore, the intermediate cylindrical portion 13, together with the insertion section 2 in the endoscope 1 inserted into the through hole 11, is bent. More specifically, the intermediate cylindrical portion 13 includes a cylindrical portion 13a having a cylindrical shape and a fin 13b protruding from an outer peripheral surface of the cylindrical portion 13a. The cylindrical portion 13a and the fin 13b are each composed of an elastically deformable resin material that is bent together with the insertion section 2. The cylindrical portion 13a is substantially the same in outer diameter throughout in the longitudinal direction.

The fin 13b has a helical shape with the longitudinal axis L used as a center axis. In other words, the fin 13b has a shape corresponding to a threaded portion of a male screw. Note that although the fin 13b is a single consecutive helix in the present embodiment as illustrated, the fin 13b may be divided into plural parts. The fin 13b may be a double helix or a triple helix.

When the insertion section 2 with the tube 10 arranged on its outer periphery is inserted into the subject, and the actuator 8 rotates the tube 10 around the longitudinal axis L, the fin 13b having a helical shape rotates around the longitudinal axis L within the subject. When the fin 13b rotates with the fin 13b abutting on an inner wall of the subject, the tube 10 applies a driving force in the distal end direction or the proximal end direction to the insertion section 2. Mobility of the insertion section 2 in the longitudinal axis direction within the subject is improved by applying the driving force.

A distal end-side opening 11a in the through hole 11 is formed in the distal end portion 14. In other words, the distal end portion 14 is a site disposed at the distal end 10a of the tube 10. The distal end portion 14 has a cylindrical shape, and includes a tapered portion 14a having an outer diameter decreasing in the distal end direction and a stiffening portion 14b provided at a distal end 14a1 of the tapered portion 14a.

The tapered portion 14a is composed of an elastically deformable resin material that is bent together with the insertion section 2. A proximal end 14a2 of the tapered portion 14a is connected to a distal end of the cylindrical portion 13a in the intermediate cylindrical portion 13, and an outer diameter at the proximal end 14a2 of the tapered portion 14a is substantially the same as an outer diameter at the distal end of the cylindrical portion 13a. An outer diameter at the distal end 14a1 of the tapered portion 14a is smaller than the outer diameter at the proximal end 14a2 of the tapered portion 14a.

An inner diameter of the tapered portion 14a, i.e., an inner diameter of the through hole 11 in the tapered portion 14a may be constant in the direction along the longitudinal axis L, may decrease in the distal end direction, or may increase in the distal end direction.

The tapered portion 14a preferably has a shape having a thickness in the direction perpendicular to the longitudinal axis L, that is, a thickness in a radial direction decreasing in the distal end direction. In the present embodiment, the inner diameter of the through hole 11 in the tapered portion 14a is made substantially constant in the direction along the longitudinal axis L, to change the thickness of the tapered portion 14a to decrease in the distal end direction.

When the thickness of the tapered portion 14a is decreased in the distal end direction, like in the present embodiment, the distal end portion 14 can be more easily deformed to match a bending shape of the insertion section 2 inserted into the through hole 11.

Since the through hole 11 is formed at the distal end 14a1 of the tapered portion 14a, the distal end 14a1 of the tapered portion 14 has an annular shape centered on the longitudinal axis L.

The stiffening portion 14b is a site protruding in the distal end direction from the distal end 14a1 of the tapered portion 14a and having an annular shape centered on the longitudinal axis L. The stiffening portion 14b is a site constituting the distal end 10a of the tube 10, and surrounds the distal end-side opening Ila in the through hole 11.

The stiffening portion 14b has a higher rigidity than a rigidity of the distal end 14a1 of the tapered portion 14a. The rigidity of the stiffening portion 14b and the rigidity of the distal end 14a1 of the tapered portion 14a each mean a hardly deformable cross-sectional shape by a plane perpendicular to the longitudinal axis L. As described above, the stiffening portion 14b and the distal end 14a1 of the tapered portion 14a are each annular in the cross-sectional shape by the plane perpendicular to the longitudinal axis L. In the present embodiment, the higher the rigidity is, the smaller an amount of deformation from an exact circle and an amount of change in inner diameter of the cross-sectional shape of the stiffening portion 14b or the distal end 14a1 having the annular shape with no stress applied become.

The rigidity of the stiffening portion 14b and the rigidity of the distal end 14a1 of the tapered portion 14a are compared with each other with both the stiffening portion 14b and the distal end 14a1 separated from each other. In other words, the rigidity of the distal end 14a1 of the tapered portion 14a means the rigidity of the distal end 14a1 of the tapered portion 14a when the stiffening portion 14b is not provided.

A configuration for making the rigidity of the stiffening portion 14b higher than the rigidity of the distal end 14a1 of the tapered portion 14a is not particularly limited. In the present embodiment, as an example, a maximum thickness of the stiffening portion 14b is larger than the thickness of the distal end 14a1 of the tapered portion 14a, as illustrated in FIG. 3. The rigidity of the stiffening portion 14b and the thickness of the distal end 14a1 of the tapered portion 14a each mean a thickness in the direction perpendicular to the longitudinal axis L, that is, in the radial direction. In the present embodiment, the stiffening portion 14b is composed of the same material as the material composing the tapered portion 14a, and is molded integrally with the tapered portion 14a.

The stiffening portion 14b in the present embodiment includes a protrusion portion 14b 1 protruding more inward in the radial direction than the distal end 14a1 of the tapered portion 14a, to have a thickness larger than the thickness of the distal end 14a1 of the tapered portion 14a, as illustrated in FIG. 3. In other words, an inner diameter of the stiffening portion 14b is smaller than the inner diameter at the distal end 14a1 of the tapered portion 14a, and an outer diameter of the stiffening portion 14b is substantially equal to or larger than the outer diameter at the distal end 14a1 of the tapered portion 14a.

When the inner diameter of the stiffening portion 14b arranged at the distal end 10a of the tube 10 is smaller than an inner diameter of a member arranged in a more proximal end direction than the stiffening portion 14b, like in the present embodiment, a lubricant with which a space between the tube 10 and the insertion section 2 is filled can be prevented from leaking out of the distal end-side opening 11a.

In the present embodiment, the protrusion portion 14b 1 in the stiffening portion 14b has a roof shape having a vertex directed inward in the radial direction. In the present embodiment, a cross-sectional shape on a plane including the longitudinal axis L of the protrusion portion 14b 1 is substantially circular or substantially triangular. Therefore, the stiffening portion 14b linearly contacts an outer peripheral surface of the insertion section 2 inserted inward.

The stiffening portion 14b is a site where the distal end-side opening 11a as an end of the through hole 11 is formed, and therefore is a site that easily contacts the insertion section 2 inserted into the through hole 11. In the present embodiment, the stiffening portion 14b has a shape linearly contacting the insertion section 2. Thus, a contact area between the stiffening portion 14b and the insertion section 2 is reduced so that a sliding resistance of the tube 10 to the insertion section 2 can be suppressed.

In the tube 10 having a configuration described above, when the stiffening portion 14b is provided at the distal end 14a1 of the tapered portion 14a in the distal end portion 14, the distal end-side opening 11a in the through hole 11 can be hardly deformable compared with a conventional tube provided with no stiffening portion 14b.

If a portion inserted into the through hole 11 in the insertion section 2 in the endoscope 1 is bent, a force in such a direction as to increase an inner diameter of the distal end-side opening 11a in the through hole 11 is applied to the tube 10. In the present embodiment, the distal end-side opening 11a is not easily deformed because the stiffening portion 14b is provided. Thus, the diameter of the distal end-side opening 11a can be more prevented from increasing than in the conventional example when the insertion section 2 is bent. Accordingly, in the tube 10 according to the present embodiment, when the diameter of the distal end-side opening 11a is more prevented from increasing than in the conventional example, the lubricant with which the space between the tube 10 and the insertion section 2 is filled can be prevented from leaking out.

In the present embodiment, when the distal end 14a1 of the tapered portion 14a in the distal end portion 14 is provided with the stiffening portion 14b, ease of deformation of the tapered portion 14a depending on the bending of the insertion section 2 can be maintained without increasing a rigidity of the tapered portion 14a itself.

As described above, in the tube 10 according to the present embodiment, when the diameter of the distal end-side opening 11a is more prevented from increasing while ease of deformation of the tapered portion 14a is maintained than in the conventional example, the lubricant can be prevented from leaking out while contact pressure between the tube 10 and the insertion section 2 is prevented from increasing. Therefore, the sliding resistance between the tube 10 and the insertion section 2 can be kept small.

### Second Embodiment

A second embodiment of the present invention will be described below. Only a difference from the first embodiment will be described below, and similar components to the components in the first embodiment are respectively assigned the same reference numerals, and description of the components is appropriately omitted.

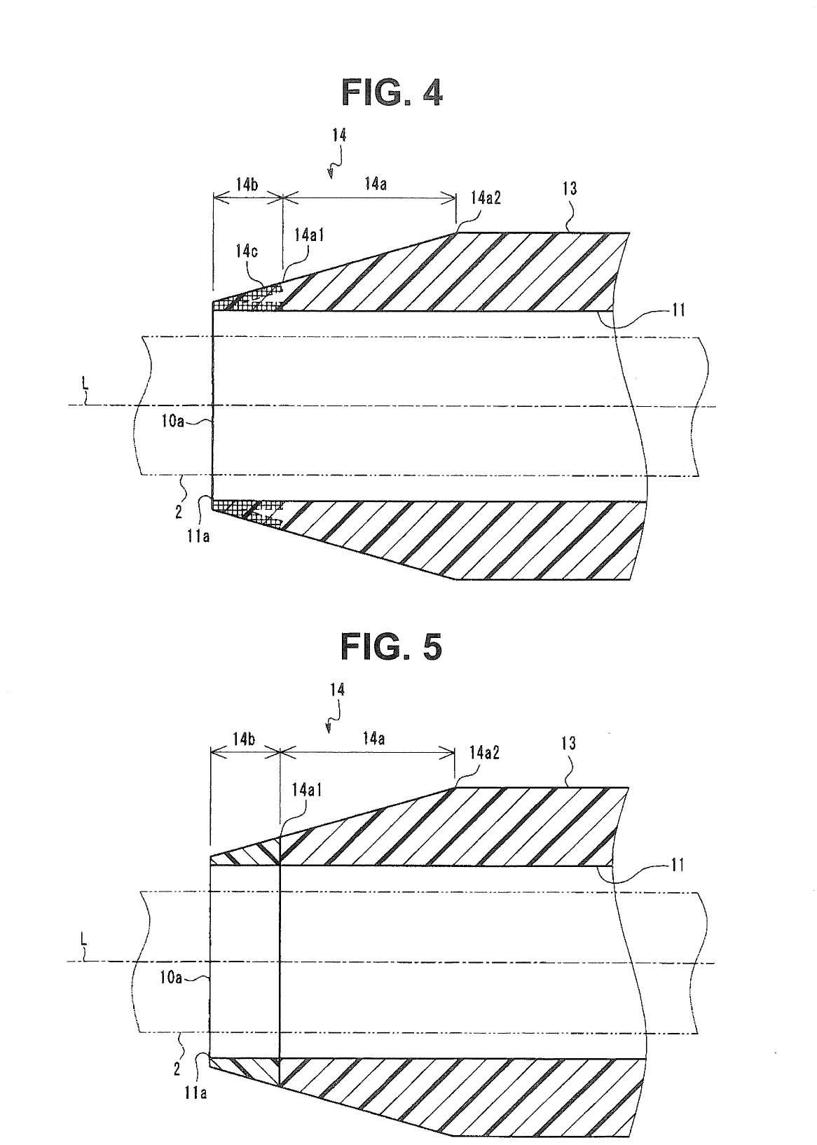

A tube 10 according to the present embodiment illustrated in FIG. 4 differs from the tube 10 according to the first embodiment in only a configuration of a stiffening portion 14b.

Although the second embodiment is the same as the first embodiment in that the stiffening portion 14b in the present embodiment is composed of the same material as a material composing a tapered portion 14a and is formed integrally with the tapered portion 14a, a rigidity of the stiffening portion 14b in the present embodiment is made higher than a rigidity of a distal end 14a1 of the tapered portion 14a by surface treatment 14c (a portion indicated by hatching in FIG. 4).

The surface treatment 14c for increasing the rigidity of the stiffening portion 14b is not particularly limited, but may increase a hardness of the stiffening portion 14b composed of a resin material. The surface treatment 14c is plating or coating, for example. The surface treatment 14c may be modification treatment for increasing the hardness of the stiffening portion 14b. As the modification treatment of the resin material, modification treatment by a medicinal solution and modification treatment by a plasma, for example, have been known.

In the tube 10 according to the present embodiment, the stiffening portion 14b is provided at the distal end 14a1 of the tapered portion 14a in a distal end portion 14, like in the first embodiment. Thus, a diameter of a distal end-side opening 11a can be more prevented from increasing while ease of deformation of the tapered portion 14a is maintained than in the conventional example, and a lubricant can be prevented from leaking out while contact pressure between the tube 10 and an insertion section 2 is prevented from increasing. Therefore, a sliding resistance between the tube 10 and the insertion section 2 can be kept small.

Note that in the present embodiment, the stiffening portion 14b may be provided with a protrusion portion protruding inward in a radial direction, like in the first embodiment.

### Third Embodiment

A third embodiment of the present invention will be described below. Only a difference from the first embodiment will be described below, and similar components to the components in the first embodiment are respectively assigned the same reference numerals, and description of the components is appropriately omitted.

A tube 10 according to the present embodiment illustrated in FIG. 5 differs from the tube 10 according to the first embodiment in only a configuration of a stiffening portion 14b.

The stiffening portion 14b in the present embodiment is composed of a second resin material different from a first resin material composing a tapered portion 14a. The second resin material composing the stiffening portion 14b has a higher hardness than a hardness of the first resin material composing the tapered portion 14a. The stiffening portion 14b in the present embodiment is composed of the material having the higher hardness than the hardness of the tapered portion 14a, and therefore has a higher rigidity than a rigidity of a distal end 14a1 of the tapered portion 14a.

A method of coupling the stiffening portion 14b and the tapered portion 14a to each other is not particularly limited. The stiffening portion 14b and the tapered portion 14a may respectively have forms to be coupled to each other by adhesion, or may respectively have forms to be coupled to each other at the time of molding by two-color molding, insertion molding, or the like.

In the tube 10 according to the present embodiment, the stiffening portion 14b is provided at the distal end 14a1 of the tapered portion 14a in a distal end portion 14, like in the first embodiment. Thus, a diameter of a distal end-side opening 11a can be more prevented from increasing while ease of deformation of the tapered portion 14a is maintained than in the conventional example, and a lubricant can be prevented from leaking out while contact pressure between the tube 10 and an insertion section 2 is prevented from increasing. Therefore, a sliding resistance between the tube 10 and the insertion section 2 can be kept small.

In the present embodiment, the stiffening portion 14b may also be provided with a protrusion portion protruding inward in a radial direction, like in the first embodiment. In the present embodiment, the stiffening portion 14b may also be subjected to surface treatment for increasing a hardness, like in the second embodiment.

The present invention is not limited to the above-described embodiments, but can be appropriately changed without departing from the gist or thought of the invention that can be understood from the claims and the entire specification, and a tube for insertion device involving such a change is also included in a technical scope of the present invention.

What is claimed is:

1. A tube body formed in a cylindrical shape extending in a longitudinal axis direction, the tube body comprising:
a tapered portion formed at at least one end portion in the longitudinal axis direction of the cylindrical shape and configured to decrease an outer diameter of the cylindrical shape toward the end portion; and
a ring-shaped reinforcement portion provided to be connected to the end portion and having a higher rigidity than a rigidity of a distal end portion of the tapered portion.

2. The tube body according to claim 1, wherein an insertion section in an endoscope formed in an elongated shape penetrates into the cylindrical shape so that the tube body is mounted on the insertion section.

3. The tube body according to claim 2, wherein an inner peripheral surface of the reinforcement portion has an inner diameter contacting an outer peripheral surface of the insertion section or opposing the outer peripheral surface with a gap.

4. The tube body according to claim 1, wherein an inner diameter of the tapered portion is set to be same as an inner diameter of a portion in the cylindrical shape other than the tapered portion, and a thickness of the tapered portion decreases toward the end portion.

5. The tube body according to claim 1, wherein a maximum thickness of the reinforcement portion is larger than a thickness at an end portion of the tapered portion.

6. The tube body according to claim 5, wherein the reinforcement portion includes a protrusion portion protruding more inward in a radial direction than the end portion of the tapered portion.

7. The tube body according to claim 6, wherein a cross-sectional shape of the protrusion portion in a plane including the longitudinal axis is a circle or a triangle having a vertex directed inward in the radial direction.

8. The tube body according to claim 1, wherein the reinforcement portion is composed of a same resin material as a resin material composing the tapered portion, and has a hardness made higher than a hardness of the tapered portion by surface treatment.

9. The tube body according to claim 1, wherein
the tapered portion is composed of a first resin material, and
the reinforcement portion is formed of a second resin material having a higher hardness than a hardness of the first resin material.

10. An endoscope comprising:
an insertion section formed in an elongated shape extending from a hand side to a distal end side in a longitudinal direction and configured to be inserted into a subject; and
a tube formed in a cylindrical shape having an inner periphery into which the insertion section penetrates and configured to be driven to rotate around an axis in the longitudinal direction with the insertion section inserted into the tube,
wherein the tube comprises
a tapered portion formed on the distal end side of the insertion section on an outer peripheral surface and having an outer diameter decreasing toward the distal end side of the insertion section, and
a reinforcement portion connected to a distal end portion of the tapered portion, formed in a ring shape having an inner peripheral surface opposing an outer peripheral surface of the insertion section to contact the outer peripheral surface or opposing the outer peripheral surface with a gap, and having a higher rigidity than a rigidity of the distal end portion of the tapered portion.
